(19) United States
(12) Patent Application Publication
(10) Pub. No.: US 2003/0023218 A1
(43) Pub. Date: Jan. 30, 2003

(54) Disposable nasal sanitary handkerchief

(76) Inventor: Eduardo Abenia, Montevideo (UY)

Correspondence Address: ABELMAN, FRAYNE & SCHWAB, 150 East 42nd Street, New York, NY, 10017, US

(21) Appl. No.: 10/150,794

(22) Filed: May 17, 2002

(30) Foreign Application Priority Data
May 18, 2001 (UY) 26,719

(51) Int. Cl. A61F 13/15

(52) U.S. Cl. 604/385.01; 604/385.19

(57) ABSTRACT

A disposable hygienic product in the form of a handkerchief, a tissue or napkin has an interior pocket formed by joining all but one of the edges of the product, the open edge being adapted to receive the user's nose. The hygienic product avoids the use of two separate objects in order to stop and contain nasal fluid. The hygienic product can be used in a fast and inexpensive way, and can substitute in certain physiological situations for the use of a handkerchief or any other element or product, with the used hygienic product being disposed of in a safe manner.

### BACKGROUND OF THE INVENTION

1. Field of the Invention

This invention is related to hygienic products, and in particular to facial and nasal tissues, napkins, handkerchiefs, and other products for sanitary purposes.

2. Description of the Related Art

In the ever-changing and dynamic industry of disposable items, many problems have been solved in order to get better results as far as bodily secretions are concerned, more specifically, nasal fluids.

Many solutions have been directed to well-known disposable paper tissues which, in different kinds of packaging, are sold worldwide. Some of these solutions with tissues have been focused on the thinness of the paper or the way in which the tissues have been packed, varying from boxes to packages made of nylon or the like. However, often such prior art tissues, napkins and handkerchiefs require a folding-over to form two separable portions for insertion of nasal fluids therebetween. After use, folded portions can open, which is not hygienic.

The ability to restrain the bodily liquid or nasal secretion and how such fluids should be treated is one of the problems that one usually has when using the regular hygienic products in the prior art, such as tissues, napkins and/or handkerchiefs.

From practical experience and tests conducted, one can establish that in specific cases, the use of such prior art hygienic products is not very practical, and such prior art hygienic products can create various drawbacks whenever one uses the products. For example, nasal irritation caused by abrasion, the nuisance and low level of hygiene which comes from touching the nasal fluid, etc.

It is evident, therefore, that the market has not found any definite solution which, as a whole, can address the problems so that a hygienic product can hold the nasal fluid but at the same time be disposable in a safe way.

As is already known, disposable goods have become an essential part of everyday life, which is why the handkerchief and hygienic product industries, with disposable tissues, have produced such handkerchiefs and hygienic products in a great quantity of different proposals which attempt to improve such products and goods. Nevertheless, and in spite of the results attained in research and evolution, such industries still cannot offer the user both an article as convenient as a handkerchief for the nasal fluids, with the handkerchief being dispensable in a container and being easily disposable.

### BRIEF SUMMARY OF THE INVENTION

To address the limitation of hygienic products of the prior art, a hygienic product is disclosed in the form of a handkerchief or other manipulable product such as a tissue or napkin which is at the same time safe and disposable. The hygienic product avoids the use of two objects in order to stop and contain nasal fluid. The hygienic product can be used in a fast and inexpensive way, and can substitute in certain physiological situations for the use of a handkerchief or any other element or product, with the used hygienic product being disposable of in a safe and sanitary manner.

### BRIEF DESCRIPTION OF THE SEVERAL VIEWS OF THE DRAWINGS

FIG. 1 shows a plan view of the disposable nasal sanitary handkerchief of the invention illustrating the manufacture of the handkerchief;

FIG. 2 shows a perspective side view of the disclosed disposable nasal sanitary handkerchief in use by a person;

FIG. 3 shows a roll of the disclosed disposable nasal sanitary handkerchief; and

FIG. 4 shows the disclosed disposable nasal sanitary handkerchief in a dispenser.

### DETAILED DESCRIPTION OF THE INVENTION

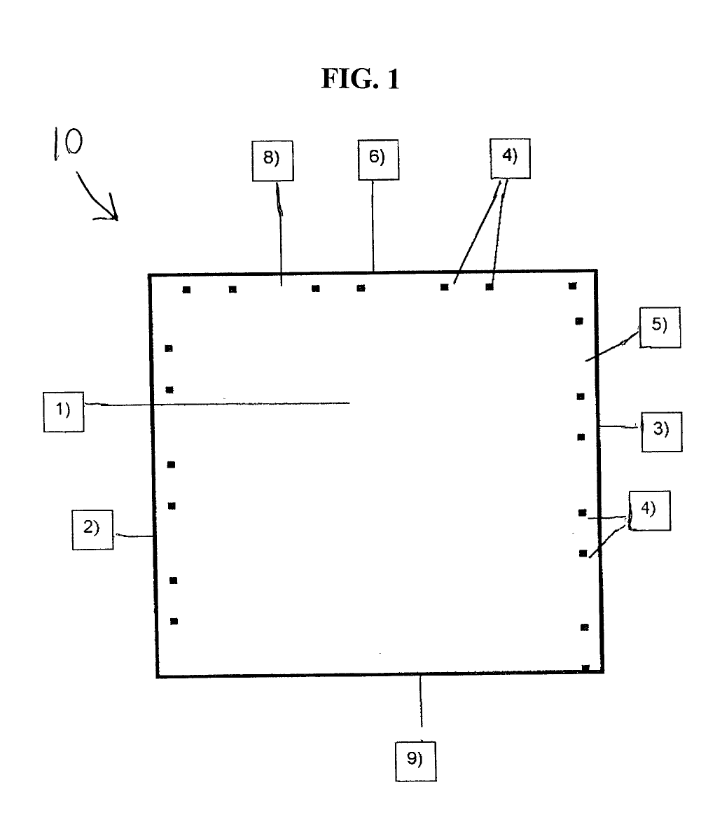

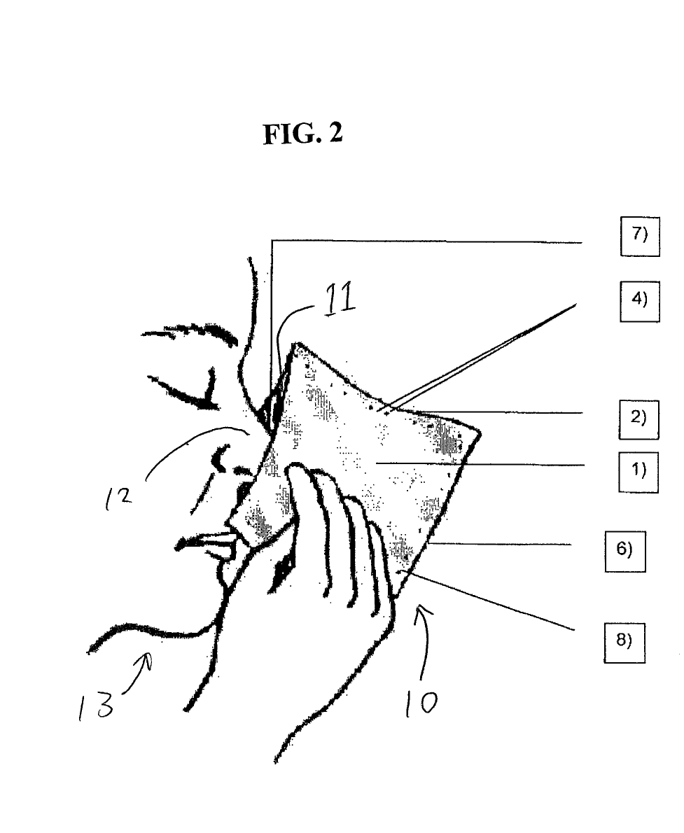

As shown in FIGS. 1-2, a hygienic disposable product 10, suitable for use with the nose, includes two layers 1, 7 made of flexible and waterproof material such as nylon, paper, or any other known flexible waterproof materials; and having, for example, four borders or edges 2, 3, 6, 9. The layers 1, 7 are positioned facing each other, and then fastened along three borders 2, 3, 6 of the four borders. The borders 2, 3, 6 of each layer are respectively fastened to each other by means of a welding 4 or other layer-fastening mechanism which allows a partial opening or gap 5, 8 between the layers 1, 7 and between pairs of weldings 4. The welding 4 may be spots of adhesive; heat seals, for example, if the layers are composed of heat-sealable materials such as plastic; or other known fastening mechanisms for joining the layers 1, 7 together.

The fourth border 9 is free of any welding or fastening mechanisms, and so allows the product 10 to open and separate apart portions of the layers 1, 7 to a sufficient extent to allow the user 13 to insert a portion of a nose 12 therein; for example, the nose 12 of the user 13, shown in FIG. 2, or the nose of another being, such as a young child, an older relative, or a pet of the user. Thus, with a nose 12 disposed in the opening at the border 9, any nasal fluids from the nose 12 can flow into the opening at the border 9 and kept in the product 10, while the partial gaps 5, 8 along the sealed borders 2, 3, 6 allow air to pass from the nose 12 and through the product 10, allowing the user 13 to blow a nose.

As shown in FIG. 2, the product 10 can include an inner layer 11 or coating of a substance. In a first embodiment of the substance, the substance can avoid or reduce irritation to the nose 12 or other skin or membranes of the user 13 caused by the mucus and other nasal fluids as a result of using the product 10 repeatedly. In a second embodiment alternative to or in conjunction with the first embodiment of the substance, the substance can absorb the nasal fluids.

The inner layer 11 of the substance can be disposed on one or more sides of the layers 1, 7 interior to the product 10. The substance of the inner layer 11 can be a gel, cold cream, aloe, an oil, and/or any known substance for preventing irritation and/or for absorbing the nasal fluids. For example, the substance of the inner layer 11 can be any gel, cream, oil, or other substance, composition, or agent described in U.S. Pat. No. 5,871,763, which is incorporated herein by reference.

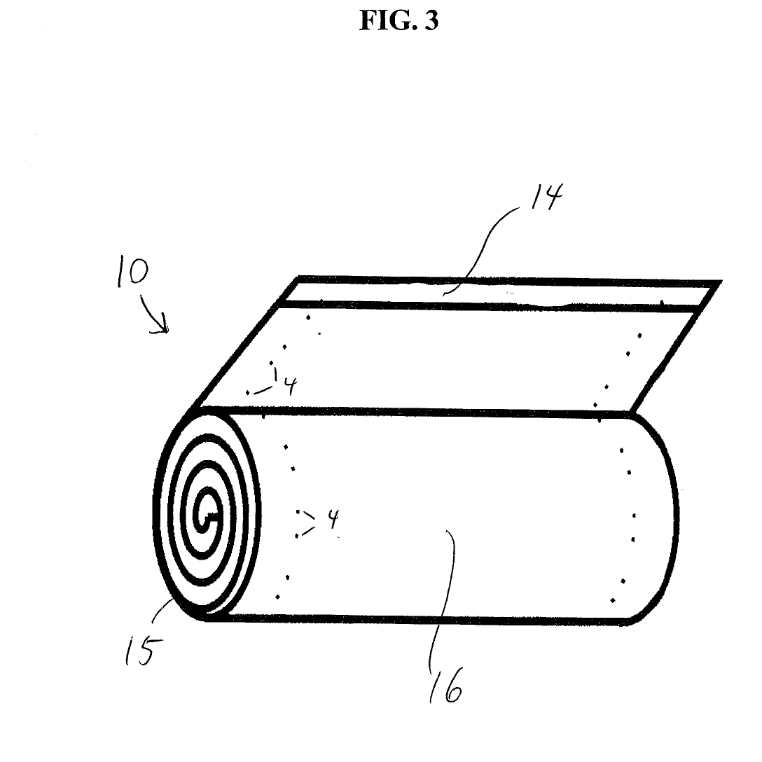

As shown in FIG. 3, the hygienic disposable product 10 can include an adhesive 14, in the form of a strip or other shapes, allowing the product 10 to be disposable, for example, to be rolled up in the form of a tubular configuration 15, allowing the product 10 after use to be readily discharged in a trash receptacle, a toilet, or other appropriate locations to dispose of the used product 10.

In an alternative embodiment, the product 10 can be dispensed to users in the form of the tubular configuration 15, with the adhesive 14 removably secured to a side 16 of the tubular configuration 15. The adhesive 14 can be composed of any known adhesive substance with sufficient adhesive characteristics to allow the product 10 to be rolled into the tubular configuration 15, to be opened up into the deployed configuration shown in FIGS. 1-2 for use by the user 13, and to be returned to the tubular configuration 15 of FIG. 3 with the adhesive 14 re-securing an end to the side 16 of the product 10 into the tubular configuration 15. Accordingly, the product 10 can be sold unused and initially in the tubular configuration 15, opened up and used, and then rolled back up to the tubular configuration 15 for disposal after use.

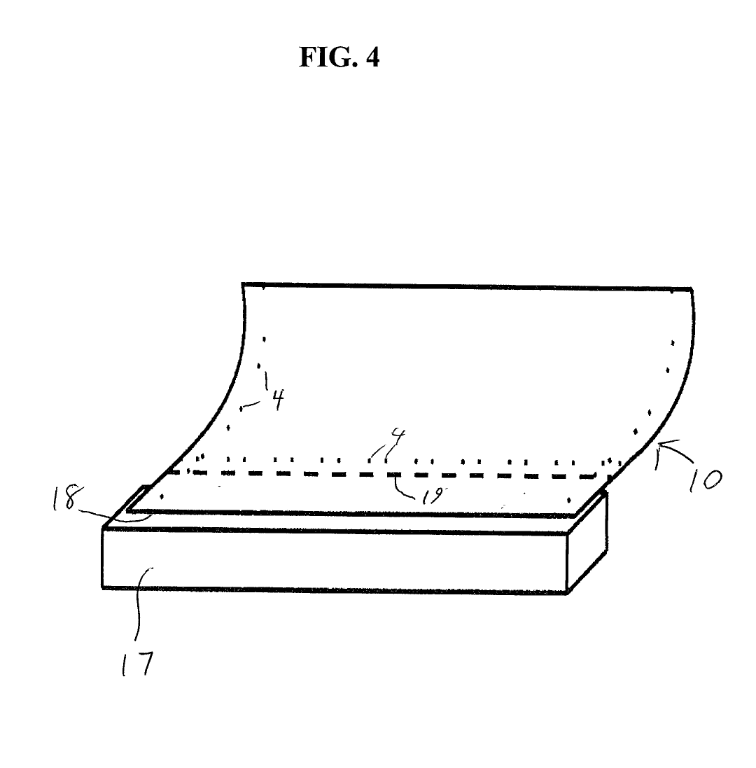

As shown in FIG. 4, the product 10 can be packaged in a dispenser 17, with a plurality of sheets of the product 10 piled in a zigzag orientation known in the art inside the dispenser 17, with a slot 18 permitting removal of individual sheets of the product 10 from the dispenser 17. The plurality of sheets of the product 10 can be connected together by a detachable seam 19, known in the art, for cutting or otherwise removing an individual sheet of the product 10 from the remaining sheets. Accordingly, the product 10 can be easier to get and remove from the dispenser.

Even though the invention has been described in exemplary embodiments in its current form, techniques can be developed which modify the embodiments of the invention. However, such modifications are included within the spirit of the invention and therefore are part of the invention as claimed herein.

What is claimed is:

1. A disposable hygienic product comprising:
two layers of material, each layer respectively having:
a first border portion; and
a plurality of second border portions, with the plurality of second border portions of each layer being joined together in at least one location on each second border portion, thereby forming a nose-insertion opening between the unattached first border portion of the layers, for receiving a nose, thereby to permit flow of nasal fluids therefrom into a pocket formed between the two layers.

2. The disposable hygienic product of claim 1, wherein the plurality of second border portions, joined together, includes:
a plurality of attachment locations on each second border portion, with air openings formed between the plurality of attachment locations for each respective second border portion, thereby allowing air expelled from the nose to pass through each respective opening.

3. The disposable hygienic product of claim 1, wherein at least one of the two layers is waterproof.

4. The product of claim 1 that is formed from a folded sheet of material.

5. The disposable hygienic product of claim 1, further comprising:
a substance disposed between the two layers for absorbing the nasal fluids.

6. The disposable hygienic product of claim 5, wherein the two layers have a respective interior side facing each other and forming an interior pocket between the two layers; and
wherein the substance is disposed on at least one interior side.

7. The disposable hygienic product of claim 1, further comprising:
a substance disposed between the two layers for reducing irritation of the nose of the user.

8. The disposable hygienic product of claim 7, wherein each of the two layers has a respective interior side facing the other and forming an interior pocket between the two layers; and
wherein the substance is disposed on at least one interior side.

9. The disposable hygienic product of claim 1, further comprising: an adhesive positioned on at least one of the layers for securing the layers in a tubular configuration for disposal.

10. The product of claim 7, where the subsance is selected from lotions, salves, gels, fragances, fluid adsorbents, disinfectants and combinations of the above.

11. The product of claim 1, wherein the material is an adsorent cellulosic facial tissue.
